(19) United States
(12) Patent Application Publication
(10) Pub. No.: US 2004/0048842 A1
(43) Pub. Date: Mar. 11, 2004

(54) Method of treating skin disorders

(76) Inventor: Kathleen McMillan, Concord, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/241,273

(22) Filed: Sep. 10, 2002

(51) Int. Cl. A61K 31/555; A61K 31/409; A61K 31/353; A61K 31/198; A61K 31/12

(52) U.S. Cl. 514/185; 514/410; 514/455; 514/561; 514/680; 604/20

(57) ABSTRACT

The invention provides a method of treating certain skin disorders, including, for example, psoriasis. Pulsed or scanned coherent or incoherent radiation, when applied to a target region, both activates a photosensitizer disposed within the region and induces selective photothermolysis of blood vessels disposed within region. The combination of photodynamic therapy and selective photothermal damage in the target region provides an effective and long lasting treatment of the skin disorder.

### FIELD OF THE INVENTION

This invention relates generally to the field of dermatology, and more particularly to a method of treating certain skin disorders by a combination of light induced photodynamic therapy and photothermal vascular damage in the treatment region.

### BACKGROUND OF THE INVENTION

T-cell mediated skin disorders afflict a significant number of individuals. For example, one commonly recognized T-cell mediated skin disorder is psoriasis, which reportedly affects more than two percent of the world's population (Robert et al. (1999) NEW ENG. J. MED. 341:1817-1828). Psoriasis is a chronic inflammatory skin condition characterized by scattered, scaly, red, cutaneous plaques that contain inflammatory infiltrates and hyperproliferative keratinocytes.

A variety of treatments for psoriasis have been developed to date. One approach involves the use of topically or systemically delivered drugs. Another approach involves the application of ultraviolet (UV) light to the psoriatic lesion (phototherapy and photochemotherapy). Other light-based approaches include selective photothermolysis and photodynamic therapy.

In the first approach, topical drugs (for example, coal tar, vitamin D₃ analogs such as calcipotriol, retinoids such as tazarotene, and corticosteroids such as hydrocortisone, dexamethasone, and betamethasone) are applied to the afflicted areas (Peters et al. (2000) AM. J. HEALTH SYST. PHARM. 57: 645-662). These treatments may produce acceptable results, however, the benefit typically is short lived. Alternatively, for patients unresponsive to topical drugs and for patients with moderate to severe disease, systemic drugs may be used (for example, methotrexate, acetretin, cyclosporin and tacrolimus (Peters et al. (2000) supra, Linden and Weinstein (1999) AM. J. MED. 107: 595-605). Adverse effects and toxicity are concerns when systemic therapy is used for psoriasis.

Furthermore, a variety of UV light-based approaches have been developed to date. Photochemotherapy involves the combination of UV radiation and drugs. For example, the drug psoralen in combination with ultraviolet A (PUVA) is commonly used. Twenty PUVA treatment sessions may be needed to achieve clearance of psoriasis (Peters et al. (2000) supra). Furthermore, this approach is associated with an increased risk of skin cancer (Stern et al. (1998) J. NATL. CANCER INST. 90: 1278-1283). Phototherapy involves use of ultraviolet B (UVB) radiation in the absence of psoralen, although phototherapy may be combined with emollients or topical drugs such as coal tar. UVB phototherapy must be administered frequently to be effective, and over long periods can lead to photoaging of the skin (Linden et al. (1999) supra).

Other light-based approaches avoid the risks associated with UV radiation. Pulsed laser sources have been used to induce selective photothermolysis of dermal vasculature in psoriatic plaques (Hacker et al. (1992) ARCH. DERMATOL. 128:853-855, Katugampola et al. (1995) BR. J. DERMATOL. 133:909-913, Ros et al. (1996) LASERS SURG. MED. 19:331-335, Bjerring et al. (1997) ACTA. DERM. VENEREOL. 77:59-61, Zelickson et al. (1996) J. AM. ACAD. DERMATOL. 35:64-68). Effective treatment was seen with treatment parameters that produced purpura as an acute side effect. Koebnerization (worsening of psoriasis triggered by stimuli including skin trauma) has not been reported as a consequence of selective photothermolysis of psoriatic plaque. Under certain circumstances, however, patients have failed to respond to the treatment or only partial clearing of the plaques occurred thereby requiring subsequent treatments. Furthermore, in some patients the level of pain limited the numbered of treatments available. In addition, photodynamic therapy has been used to treat psoriatic lesions in individuals afflicted with psoriasis (Boehncke et al. (2000) ARCH. DERMATOL. 136:271-272, Ceburkov et al. (2000) EUR. J. DERMATOL. 10:568-576, Collins et al. (1997) BR. J. DERMATOL. 137: 743-749, Ibbotson (2002) BR. J. DERMATOL. 146: 178-188). Under certain circumstances, the patients failed to respond to the treatment, or when improvements were noted relapses occurred thereby requiring subsequent treatments. Furthermore, in certain photodynamic therapy treatments, Koebnerization was noted (Stender et al. (1996) ACTA. DERMAT. VENEREOL 76: 392-393).

Notwithstanding the foregoing, there is still an ongoing need for other methods of treating T-cell mediated skin disorders that are more effective and/or long lasting than the currently available methods.

### SUMMARY OF THE INVENTION

A variety of methods have been developed to date for the treatment of certain skin disorders. However, the existing methods typically have undesirable side effects and/or need to be repeated on occasion to achieve a satisfactory treatment. The method described herein is based upon the combined, preferably simultaneous, treatment of a skin disorder via photodynamic therapy and selective photothermolysis of blood vessels in a targeted region of the skin disorder to provide an effective, long lasting treatment of the skin disorder. In other words, the invention provides a treatment method containing two components, a photodynamic reaction and a photothermolysis reaction, both of which act together to produce a more effective and long lasting treatment.

In one aspect, the invention provides a method of treating a T-cell mediated skin disorder (for example, psoriasis, atopic dermatitis, mycosis fungoides, or lichen planus) in a pre-selected region of a mammal, preferably, a human. The method comprises administering to the mammal an amount of a photosensitizer or a pro-photosensitizer sufficient to permit an effective amount of photosensitizer to localize within the target region. Once an effective amount of photosensitizer is present, a beam of pulsed or scanned electromagnetic radiation is delivered to the target region so as to activate the photosensitizer. Once activated, the photosensitizer produces free radicals or other reactive species, for example, reactive oxygen species (for example, singlet oxygen) which when produced induce localized damage to the surrounding tissue and vasculature.

In addition to activating the photosensitizer, the beam of pulsed or scanned electromagnetic radiation also causes specific photothermolysis of blood vessels in the target region. For this effect, the beam of pulsed electromagnetic radiation is absorbed by blood or blood components in the target region. When absorbed, the electromagnetic radiation causes localized heating and lysis of the blood vessels in the target region.

In one embodiment, the photosensitizer can be a photoreactive chromophore useful in photodynamic therapy. Useful photosensitizers include, for example, chlorins, cyanines, purpurins and porphyrins, for example, benzoporphyrin derivative monoacid (BPD-MA) and hematoporphyrin derivative (HPD). Other useful photosensitizers include, for example, bacteriochlorins and bacteriopurpurins, for example, 5,10-octaethylbacteriopurpurin. Still other useful photosensitizers include xanthenes, for example, rose bengal, or naturally occurring photosensitizers, for example, hypericin. These photosensitizers may be administered systemically (for example, intravenously or orally), or topically (for example, applied directly to the region of interest).

In another embodiment, the photosensitizer can be produced following administration of a pro-photosensitizer that is metabolized or converted in the recipient to generate the photosensitizer, or that induces the synthesis of the photosensitizer. The photosensitizer may be generated in the target area or may be generated elsewhere in the recipient and then transported to the target area. Pro-photosensitizers useful in the practice of the invention include, for example, 5-aminolevulinic acid (ALA) and derivatives of ALA, for example, ALA-methyl ester, ALA-n-pentyl ester, ALA-n-octyl ester, R,S-ALA-2-(hydroxymethyl)tetrahydropyranyl ester, N-acetyl-ALA, and N-acetyl-ALA-ethyl ester. The pro-photosensitizers may be administered systemically (for example, intravenously, or orally) or topically (for example, applied directly to the region of interest).

The pulsed or scanned beam of electromagnetic radiation may be either incoherent or coherent light. Coherent light sources, for example, pulsed dye lasers, are preferred. The beam of pulsed coherent radiation can be a beam of pulsed laser radiation of wavelength from 460 nanometers to 620 nanometers, fluence from 1 to 120 joules per square centimeter per pulse, pulse duration from about 1 microsecond to 100 milliseconds per pulse, and spot size from 1 millimeter to 30 millimeters in diameter.

The beam of pulsed or scanned laser radiation optionally has a wavelength ranging from about 500 nanometers to about 610 nanometers. Optionally, the laser radiation has a wavelength ranging from about 580 nanometers to about 600 nanometers. Optionally, the laser radiation has a wavelength ranging from about 585 nanometers to about 595 nanometers. With regard to fluence, the laser radiation optionally has a fluence from about 2 to about 90 joules per square centimeter per pulse, optionally in the range of 2 to 30 joules per square centimeter per pulse, or optionally in the range of 4 to 20 joules per square centimeter per pulse.

The beam of pulsed or scanned radiation can have a pulse duration in the range from about 10 microseconds to about 20 milliseconds per pulse. Optionally, the laser radiation can have a pulse duration in the range from about 100 microseconds to about 10 milliseconds per pulse. Also, the beam of pulsed or scanned laser radiation can have a spot size from about 5 millimeters to about 20 millimeters in diameter.

The parameters of the pulsed or scanned beam of electromagnetic radiation should be sufficient to induce photothermolysis of blood vessels and create purpura in the region of interest, even when photosensitizer is absent. Furthermore, the method of the invention preferably is more effective when both the photodynamic reaction and photothermolysis reactions are combined relative to either a photodynamic reaction alone (e.g., where a subpurpuric dose of light is used to activate the photosensitizer) or a photothermolysis reaction alone (e.g., when little or no photosensitizer or pro-photosensitizer is present in the target region).

The treatment results in the reduction of one or more of (i) the surface area of the lesion, (ii) the thickness of the lesion, (iii) the coloration, for example, redness, of the lesion, and (iv) the amount of scaling at the site of the lesion. Although the method produces a long lasting effect and, therefore, reduces the number of treatments necessary, it is contemplated that the process may be repeated, as and when desired.

In another aspect, the invention provides a method of treating a psoriatic lesion, for example, a psoriatic plaque, in a pre-selected region of a mammal, for example, a human. The method comprises administering to the mammal, an amount of a photosensitizer or a pro-photosensitizer sufficient to permit an effective amount of photosensitizer to localize within the region. Once present in an effective amount at the region, a beam of pulsed or scanned laser radiation is delivered to the region. The beam of laser radiation is applied in an amount and for a time sufficient to (i) induce selective photothermolysis of blood vessels disposed within the region and (ii) activate the photosensitizer in the region. As a result, the method ameliorates one or more symptoms of the psoriatic lesion including, for example, (i) the surface area of the lesion, (ii) the thickness of the lesion, (iii) the coloration, for example, the redness, of the lesion, and/or (iv) the amount of scaling at the site of the lesion.

In one embodiment, the photosensitizer can be a photoreactive chromophore useful in photodynamic therapy. Useful photosensitizers include those listed herein, and may be administered systemically (for example, intravenously or orally) or topically (for example, applied directly to the region of interest).

In another embodiment, the photosensitizer can be produced following administration of a pro-photosensitizer that is metabolized or converted in the recipient to generate the photosensitizer, or that induces the synthesis of the photosensitizer. The photosensitizer may be generated in the target area or may be generated elsewhere in the recipient and then transported to the target area. Pro-photosensitizers useful in the practice of the invention include those listed herein, and may be administered systemically (for example, intravenously or orally) or topically (for example, applied directly to the region of interest).

The pulsed or scanned beam of electromagnetic radiation may be either incoherent or coherent light. Coherent light sources, for example, pulsed dye lasers, are preferred. The beam of pulsed coherent radiation can be a beam of pulsed laser radiation of wavelength from 460 nanometers to 620 nanometers, fluence from 1 to 120 joules per square centimeter per pulse, pulse duration from about 1 microsecond to 100 milliseconds per pulse, and spot size from 1 millimeter to 30 millimeters in diameter.

The beam of pulsed or scanned laser radiation optionally has a wavelength ranging from about 500 nanometers to about 610 nanometers. Optionally, the laser radiation has a wavelength ranging from about 580 nanometers to about 600 nanometers. Optionally, the laser radiation has a wavelength ranging from about 585 nanometers to about 595 nanometers. With regards to fluence, the laser radiation optionally has a fluence from about 2 to about 90 joules per square centimeter per pulse, or optionally in the range of 2 to 30 joules per square centimeter per pulse, or optionally in the range of 4 to 20 joules per square centimeter per pulse.

The beam of pulsed or scanned radiation can have a pulse duration in the range from about 10 microseconds to about 20 milliseconds per pulse. Optionally, the laser radiation can have a pulse duration in the range from about 100 microseconds to about 10 milliseconds per pulse. Also, optionally, the beam of pulsed or scanned laser radiation can have a spot size from about 5 millimeters to about 20 millimeters in diameter.

The parameters of the pulsed or scanned beam of electromagnetic radiation should be sufficient to induce photothermolysis of blood vessels and create purpura in the target region, even when photosensitizer is absent. Furthermore, the method of the invention preferably is more effective when both the photodynamic reaction and photothermolysis reactions are combined relative to either a photodynamic reaction alone or a photothermolysis reaction alone.

Although the method produces a long lasting effect and, therefore, reduces the number of treatments necessary, it is contemplated that the process may be repeated, as and when desired. In addition, under certain circumstances, the effectiveness of the treatment may be improved by removing scales from the psoriatic lesion prior to administration, for example, by topical application, of the photosensitizer or the pro-photosensitizer. Furthermore, under certain circumstances, the effectiveness of treatment may be improved by removing scales from the psoriatic plaque after administration, for example, by systemic administration, of the photosensitizer or the pro-photosensitizer, but prior to irradiation. The psoriatic scales may be removed by a physical process (for example, by abrasion), by a chemical process (for example, by application of a descaling agent, for example, salicylic acid, to the lesion, or by administration of a drug, for example, a topical drug, for example, calcipotriol, to the lesion), or by a combination of physical and chemical processes.

### BRIEF DESCRIPTION OF THE DRAWINGS

The objects and features of the invention may be more fully understood by reference to the following drawings, in which:

FIG. 1 is a graph showing the absorption bands of protoporphyrin IX produced as a result of 5-aminolevulinic acid metabolism.

FIG. 2 is a graph showing the visible absorption spectra of oxyhemoglobin (solid line) and deoxyhemoglobin (dotted line).

### DETAILED DESCRIPTION OF THE INVENTION

The invention provides a method of treating certain T-cell mediated skin disorders. The method involves the application of pulsed or scanned coherent and/or incoherent radiation to a region afflicted with the T-cell mediated skin disorder. The radiation is applied to the target region to activate a photosensitizer present in the region to initiate a photodynamic reaction. However, the rate at which the radiation is applied is greater than that necessary to activate the photosensitizer. When absorbed by blood or blood components in the region, the radiation also induces a selective photothermal reaction, more specifically, selective photothermolysis of blood vessels within the region.

The method, therefore, comprises administering to a mammal afflicted with T-cell mediated skin disorder, an amount of a photosensitizer or a pro-photosensitizer sufficient to permit an effective amount of photosensitizer to localize within a target region afflicted by with the skin disorder. Afterwards, pulsed or scanned coherent or incoherent radiation is applied to the target region to (i) activate the photosensitizer for a photodynamic therapy in the target region, and (ii) cause selective photothermolysis of blood vessels disposed within the target region.

As used herein, the term “T-cell mediated skin disorder” means a skin disorder in which T-cells participate in the pathogenesis of the disorder. Specific T-cell mediated skin disorders include, for example, psoriasis, parapsoriasis, cutaneous graft-versus host disease, dermatitis, for example, atopic dermatitis, and allergic contact dermatitis, and cutaneous T-cell lymphoma, for example, mycosis fungoides, and lichen planus. For purposes of this invention, however, it is understood that actinic keratosis is not a T-cell mediated skin disorder.

The following describes in more detail the various photosensitizers and pro-photosensitizers useful in the practice of the invention together with their modes of administration. In addition, the following describes methods for both activating the photosensitizers and inducing selective photothermolysis of blood vessels in the target region.

Photosensitizer Considerations

As used herein, the term “photosensitizer” means a photoactive chromophore that can be used in photodynamic therapy. Photosensitizers useful in the practice of the invention are activated by pulsed or scanned coherent or incoherent electromagnetic radiation having a wavelength in the range from about 460 nanometers to about 620 nanometers. As will be discussed in more detail below, the parameters of the coherent or incoherent electromagnetic radiation preferably are selected so that the radiation is capable of (i) penetrating the skin to a certain depth, (ii) activating the photosensitizer, and (iii) inducing selective photothermolysis of blood vessels in the tissue.

Photosensitizers useful in the practice of the invention include, for example, chlorins, cyanines, purpurins and porphyrins, for example, benzoporphyrin derivative monoacid (BPDMA) (available from QLT, Inc., Vancouver, Canada). Other useful photosensitizers include, for example, bacteriochlorins and bacteriopurpurins, such as those described in U.S. Pat. No. 6,376,483 B1, for example 5, 10-octaethylbacteriopurpurin, and 5, 15-octaethylbacteriopurpurin, or nickel 5, 10-bis-acrylate etioporphyrin I. Other useful photosensitizers include xanthenes, for example, rose bengal, or other photosensitizers that may be isolated or derived from natural sources, or synthesized de novo, for example, hypericin (available from Sigma Chemical Co., St. Louis, Mo.). It is understood that this list of photosensitizers including those in Table I is exemplary, and that other photosensitizers currently available or yet to be developed having the appropriate spectral characteristics may also be useful in the practice of the invention.

As used herein, the term “pro-photosensitizer” means any molecule, which when administered to a mammal is capable of being metabolized or otherwise converted to produce a photosensitizer, or is capable of stimulating the synthesis of an endogenous photosensitizer. It is contemplated that the pro-photosensitizer may be converted into a photosensitizer of interest or stimulate the synthesis of an endogenous photosensitizer at the site of the skin lesion. Alternatively, the pro-photosensitizer may be converted into a photosensitizer or stimulate the synthesis of an endogenous photosensitizer at a region remote from the skin lesion, after which the photosensitizer is transported to the skin lesion, for example, via the vasculature.

It is contemplated that pro-photosensitizers useful in the practice of the invention include, for example, precursors of protoporphyrin IX, for example, 5-aminolevulinic acid (ALA) (available from Sigma Chemical Co., St. Louis, Mo.), ALA derivatives, such as, ALA-methyl ester, ALA-n-pentyl ester, ALA-n-octyl ester, R,S-ALA-2-(hydroxymethyl)tetrahydropyranyl ester, N-acetyl-ALA, N-acetyl-ALA-ethyl ester, and those described in U.S. Pat. No. 6,034,267.

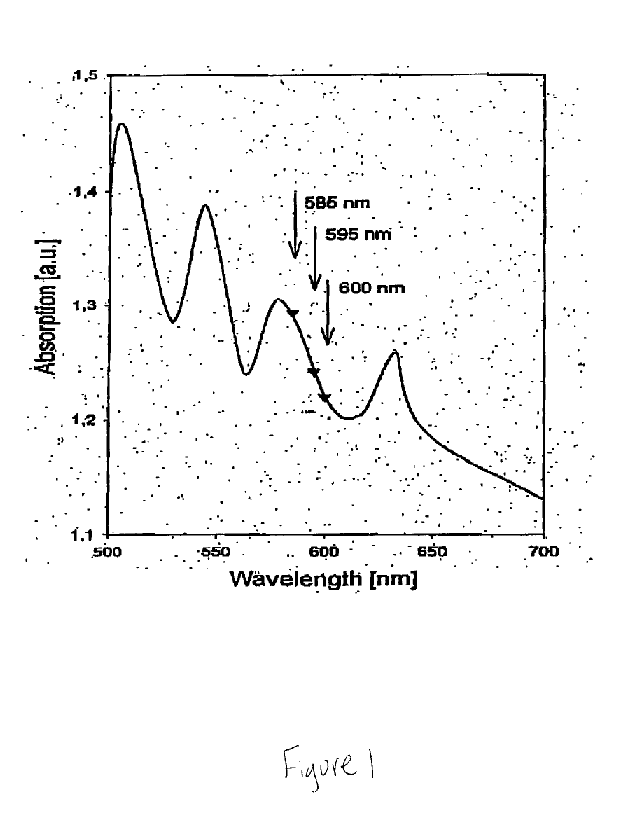

FIG. 1 is a graph showing the absorption bands of protoporphyrin IX, which is produced as a result of 5-aminolevulinic acid metabolism in a mammal. Protoporphyrin IX has an absorption band located in the blue spectral region (the 410 nanometer Soret band) and other weaker absorption bands in the 500 to 650 nanometer visible region, namely at about 506 nanometers, 546 nanometers, and 578 nanometers. In particular, protoporphyrin IX absorbs light having a wavelength of 585 to 595 nanometers and, therefore, as discussed below, may be activated by coherent or incoherent radiation that can also induce photothermolysis of blood vessels in the dermal layer of the skin.

Table 1 lists several photosensitizers useful in the practice of the invention, and identifies relevant absorption peaks for each of the listed photosensitizers.

TABLE 1

| | Wavelength of absorption |
|---|---|
| Photosensitizer | peaks (nm) |
| 5,10-octaethylbacteriopurpurin | 563, 598 |
| 5,15-octaethylbacteriopurpurin | 558, 592 |
| nickel 5,10-bis-acrylate | 580 |
| etioporphyrin I | |
| protoporphyrin IX | 506, 546, 578 |
| benzoporphyrin derivative | ˜585 |
| mono-acid | |
| hypericin | 550, 595 |
| rose bengal | 548 |
| hematoporphyrin derivative | 505, 537, 565 |

It is considered that the choice of the appropriate photosensitizer or pro-photosensitizer, formulation, dosage, and mode of administration will vary depending upon several factors including, for example, the skin disorder to be treated, and the age, sex, weight, and size of the mammal to be treated, and may be varied or adjusted according to choice. The photosensitizer or pro-photosensitizer is administered so as to permit an effective amount of photosensitizer to be present in the target region. As used herein, the term “effective amount” means an amount of photosensitizer suitable for photodynamic therapy, i.e., the photosensitizer is present in an amount sufficient to produce a desired photodynamic reaction at the target site. The photosensitizer or prophotosensitizer may be administered in a single dose or multiple doses over a period of time to permit an effective amount of photosensitizer to accumulate in the target region. Fluorescence spectroscopy or other optical detection or imaging techniques may be used to determine whether photosensitizer is present in the target region.

The photosensitizers or pro-photosensitizers may be formulated into delivery systems that deliver or permit the accumulation of photosensitizer to the target tissue. Such formulations may include coupling the photosensitizer to a specific binding ligand which binds to a target in the tissue of interest, and/or by formulation with a carrier that can deliver the photosensitizer or prophotosensitizer to the target tissue.

In addition, the compositions of the invention may be formulated in conventional manner with one or more physiologically acceptable carriers or excipients, according to techniques well known in the art. Compositions may be administered topically, orally or systemically. Under certain circumstances and depending upon the photosensitizer and/or the pro-photosensitizer chosen, topical compositions may be preferred. Topical compositions may include liposomal formulations, emulsions, gels, creams, ointments, sprays, lotions, salves, sticks, soaps, powders, aerosols, drops and any of the other conventional pharmaceutical forms in the art. Ointments and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or coloring agents. Powders may be formed with the aid of any suitable powder base. Drops may be formulated with an aqueous or non-aqueous base also comprising one or more dispersing, solubilizing or suspending agents. Aerosol sprays are conveniently delivered from pressurized packs, with the use of a suitable propellant. In addition, the photosensitizer or pro-photosensitizer may be administered topically using an external energy source, for example, by electrical means (for example, by iontophoresis) or by ultrasound (for example, by therapeutic ultrasound).

Alternatively, the photosensitizer or pro-photosensitizer may be provided in a form adapted for oral, or parenteral administration, for example, by intramuscular, intradermal, subcutaneous, intraperitoneal or intravenous injection. Alternative pharmaceutical forms thus include plain or coated tablets, capsules, suspensions and solutions containing the active component optionally together with one or more inert conventional carriers and/or diluents, for example, with corn starch, lactose, sucrose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat or suitable mixtures thereof.

In a preferred embodiment, the pro-photosensitizer, 5-aminolevulinic acid is useful in the practice of the invention. This pro-photosensitizer has been used in the photodynamic treatment of psoriatic plaques (see, for example, U.S. Pat. No. 5,079,262, Collins et al. (1997) BR. J. DERMATOL. 137: 743-749, Boehncke & Elshorst-Schmidt (2000) ARCH DERMATOL. 136: 271272, Ibbotson (2002) BR. J. DERMATOL. 146: 178-188). 5-aminolevulinic acid is a precursor in the synthesis of protoporphyrin IX, a naturally occurring photosensitizer, which itself is a precursor in the synthesis of heme (see, U.S. Pat. Nos. 5,079,262, 5,211,938, and 5,955,490). Both 5-aminolevulinic acid and protoporphyrin IX are normal products of metabolism and, therefore, in general, induce few or no side-effects. It is believed that all nucleated cells have at least a minimal capacity to synthesize protoporphyrin IX. Typically, the synthesis of protoporphyrin IX is regulated so that it is produced in cells at a rate just sufficient satisfy the need for heme. Although the synthesis of 5-aminolevulinic acid is a rate-limiting step in the synthesis of heme, it is believed this step can be bypassed by providing exogenous 5-aminolevulinic acid, or other precursors of protoporphrin IX.

5-aminolevulinic acid is an effective inducer of protoporphyrin IX when given orally, topically, or by injection (see, U.S. Pat. Nos. 5,079,262, 5,211,938, and 5,955,490). The oral and parenteral routes lead to the induction of clinically useful concentrations of protoporphyrin IX in the skin. Furthermore, because protoporphyrin IX apparently can be synthesized in the skin, 5-aminolevulinic acid may be applied topically to the target region. For dermatological applications, 5-aminolevulinic acid usually is administered topically.

It is contemplated that 5-aminolevulinic acid may be applied topically as an ointment containing from about 1% to about 40%, more preferably from about 5% to about 30%, and most preferably from about 10% to about 20% (wt/wt) 5-aminolevulinic acid in a suitable pharmaceutical acceptable carrier or excipient. The typical formulation may comprise a solution, emulsion, cream, or liposomal formulation. Furthermore, 5-aminolevulinic acid may be delivered iontophoretically to the surface of the skin (see, Rhodes et al. (1997) J. INVEST. DERMATOL. 108: 87-91). Alternatively, 5-aminolevulinic acid may be administered orally in solution, for example, fruit juice, at a final dosage of about 1 to about 60 mg/kg of body weight, at a dosage of about 5 to about 40 mg/kg of body weight, or at a dosage of about 10 to about 20 mg/kg of body weight.

It should be noted that the various parameters used for photodynamic therapy and/or selective photothermolysis are interrelated. Therefore, the photosensitizer or pro-photosensitizer dosage should be adjusted with respect to the irradiation parameters, including, for example, wavelength, fluence, irradiance, duration of the light, and the time interval between administration of the photosensitizer or pro-photosensitizer and the irradiation, and the cooling parameters, if surface cooling is desired. All of these parameters should be adjusted to produce both a photodynamic reaction resulting from activation of the photosensitizer in the target region, and a selective photothermal effect, for example, selective photothermolysis of blood vessels in the target region.

Following administration, the area treated is exposed to light to achieve the photodynamic effect. The length of time following administration, at which the light exposure takes place will depend on the nature of the composition, and the mode of administration. This time can range from about 0.1 to 48 hours post administration, more preferably in the range from 0.25 to 25 hours and, most preferably in the range from 0.50 to 15 hours.

Irradiation Considerations

It is understood that the coherent or incoherent radiation should be capable of penetrating the skin and both activating the photosensitizer present in the region of irradiation and inducing photothermolysis of blood vessels in the same region. Selective photothermolysis occurs when a chromophore in the target tissue absorbs light and the pulse duration of the light is shorter than or approximately equal to the thermal relaxation time of the target tissue (Anderson & Parrish (1983) SCIENCE 220: 324). In addition to the requirement that the wavelength of the light be absorbed by blood or blood components, the pulse duration or exposure time should be short enough such that the heat is confined primarily in the blood vessels during the radiation pulse. Consequently, irreversible thermal damage may be achieved with little or no damage to surrounding unpigmented tissue structures.

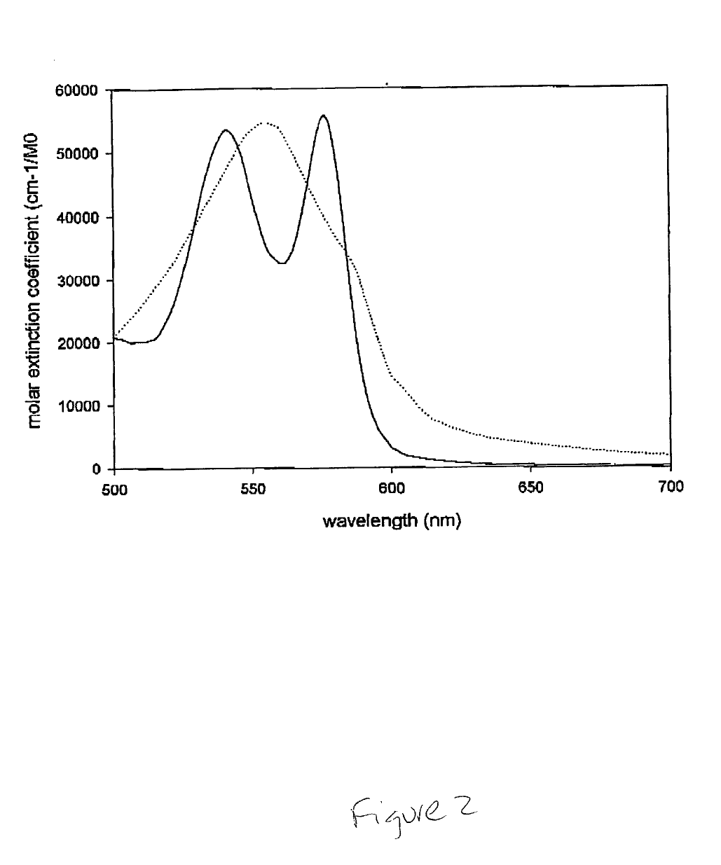

In order to achieve photothermolysis, it is understood that sufficient light energy must be absorbed by blood or blood components in the region to cause localized heating. FIG. 2 shows a graph showing the visible absorption spectra of oxyhemoglobin (solid line) and deoxyhemoglobin (dotted line), the major light absorbing components of blood. Hemoglobin has absorption peaks at about 542 nanometers and about 576 nanometers. Deoxyhemoglobulin has a broad absorption peak with maximal absorption at about 556 nanometers. The desired radiation preferably has a wavelength that is absorbed by hemoglobin and/or deoxyhemoglobin. Furthermore, the wavelength of the coherent or incoherent radiation is of some importance, as it has been shown that between 1 and 10 percent of incident red light (600-700 nm) can pass through a slab of human tissue 1 cm thick, whereas only 0.001 percent or less of blue light (about 400 nm) can pass through the same thickness of human tissue (U.S. Pat. No. 5,079,262).

By way of example, Table 2 lists the estimated skin penetration depth of light of differing wavelengths.

TABLE 2

| | Estimated penetration depth |
|---|---|
| Wavelength (nm) | (mm)¹ |
| 440 | 0.6 |
| 460 | 1.0 |
| 480 | 1.3 |
| 500 | 1.6 |
| 520 | 1.7 |
| 540 | 1.2 |
| 560 | 1.7 |
| 580 | 1.4 |
| 600 | 4.2 |
| 620 | 5.2 |
| 640 | 5.8 |
| 660 | 6.3 |

¹The penetration depths were estimated by the method of Jacques (1992) PROC. SPIE 1645: 155-165 assuming skin with 1% volume fraction blood.

According, the wavelength of irradiation for inducing photothermolysis of blood vessels preferably is in the range from about 460 nanometers to about 620 nanometers, optionally in the range from about 500 nanometers to 610 nanometers, optionally in the range from about 580 nanometers to 600 nanometers, or optionally in the range of 585 nanometers to 595 nanometers. In the range of 500 to 600 nanometers, selectively for blood vessels, preferably microvessels, is high. However, under certain circumstances it may be advantageous to use wavelengths greater than about 500 nanometers, where melanin absorption by epidermis is reduced. Radiation is the 580 nanometer to 600 nanometer region is well known to induce selective photothermolysis of blood vessels in the dermis that constitute benign vascular lesions such as port wine stain birthmarks.

Suitable light sources useful in the practice of the invention include (i) incoherent light sources optionally with one or more light filters, and (ii) coherent light sources. Coherent light sources are preferred. Suitable incoherent light sources include, for example, flash lamps, and filtered flash lamps. Suitable coherent light sources include, for example, argon lasers, argon pumped dye lasers, frequently doubled Neodymium YAG lasers, and a flash lamp-pulsed dye laser (for example, the 585 nm light emitting flash lamp-pulsed dye laser (C beam from Candela Corp., Wayland, Mass.), and the 595 nm light emitting flash lamp-pulsed dye laser (V beam from Candela Corp., Wayland, Mass.).

While the 460 nanometer to 620 nanometer wavelength range includes the relatively strong absorption peaks of oxyhemoglobin and deoxyhemoglobin, these blood components have other absorption peaks (although of lower relative maxima) at longer wavelengths. For example, deoxyhemoglobin has a weaker absorption peak at about 757 nanometers, and oxyhemoglobin has a weaker absorption peak at about 920 nanometers. Pulsed light sources in the red and near infrared (up to approximately 940 nanometers), for example, the flash lamp pumped alexandrite laser (755 nanometer), or semiconductor diode lasers, for example, gallium arsenide laser (810 nanometer) are capable of selective photothermolysis of blood vessels, including the microvessels in psoriatic lesions. Accordingly, under certain circumstances, it may be advantageous to use a source of pulsed or scanned irradiation having a wavelength in the range from about 640 nanometers to about 940 nanometers to perform combined selective photothermolysis of blood vessels and photodynamic therapy (using the appropriate photosensitizer) in the target region. Useful photosensitizers that absorb light in this spectral region include, for example, Pd(II)-octabutoxyphthalocyanine (732 nm, 838 nm), Si(IV)-napthalocyanine (773 nm)(Jori (1996) J. PHOTOCHEM. PHOTOBIOL. B 87-93), and 5,15-etiobacteriopurpurin (768 nm, 843 nm) (U.S. Pat. No. 6,376,483 B1).

As the various light parameters are interrelated, suitable light parameters should be chosen to achieve photothermolysis of blood vessels, preferably, resulting in purpura in the target region. Purpura is indicative of vascular coagulation and extravasation, and requires a localized and transient temperature in the range of about 60° C. to about 100° C.

In addition to the preferred wavelength ranges discussed above, the beam of coherent or incoherent light has a fluence in the range from about 1 to about 120 joules per square centimeter per pulse. Optionally, the laser radiation has a fluence in the range from about 2 to about 90 joules per square centimeter per pulse, optionally in the range of from about 2 to about 30 joules per square centimeter per pulse, or optionally in the range from about 4 to about 20 joules per square centimeter per pulse. In a preferred embodiment, when the radiation has a wavelength in the range of 585 to 595 nanometers, the fluence is in the range of from about 2 to about 30 joules per square centimeter per pulse.

In addition, the coherent or incoherent light has a pulse duration in the range from about 1 microsecond to about 100 milliseconds per pulse, optionally in the range from about 10 microseconds to about 20 milliseconds per pulse, or optionally in the range from about 100 microseconds to about 10 milliseconds per pulse. Typically, pulsed light sources used for targeting microvessels in skin lesions have pulse durations, or in the case of scanned continuous wave lasers have exposure times, in the range of hundreds of microseconds to tens of milliseconds. In some cases, longer pulse durations that consist of a series of shorter micropulses may be used to selectively target microvessels.

For the specific case of psoriatic plaque, the pulse duration of the light source should be shorter than or approximately equal to the thermal relaxation time of the therapeutic target, which is the microvasculature of the superficial vascular plexus including the capillary loops implicated in lymphocyte extravasation. The outer diameter of intrapapillary capillary loops in psoriatic plaque have been found to be up to 30 microns in diameter (Braverman (1997) MICROCIRCUL. 4:329-336). As a result, an appropriate pulse duration or exposure time for photothermal vascular targeting of psoriatic lesions is in the range of about 200 microseconds to about 1 millisecond. Longer pulses that consist of multiple micropulses each on the order of tens or hundreds of microseconds are also appropriate for targeting psoriatic microvasculature.

In addition, the coherent or incoherent light has a spot size in the range from about 1 millimeter to about 30 millimeters in diameter, or optionally in the range from about 5 millimeters to about 20 millimeters in diameter.

Exemplary treatment conditions sufficient to induce photothermolysis or partial photothermolysis of blood vessels are described, for example, in Bjerring et al. (1997) ACTA. DERM. VENEREOL. 77: 59-61; Katugampola et al. (1995) BR. J. DERMATOL. 133: 909-913; Zelickson et al. (1996) J. AM. ACAD. DERMATOL. 35: 64-68; Hacker & Rasmussen (1992) ARCH. DERMATOL. 128: 853-855.

Surface Cooling

Under certain circumstances, it may be advantageous to minimize thermal injury to the epidermis and upper layers of the dermis during the procedure. This may be accomplished by cooling the skin surface prior to, contemporaneous with, and/or after irradiation. Also, it is contemplated that the cooling can be applied at intervals between the pulses of irradiation.

Cooling may be facilitated by one or more cooling systems known and used in the art. Cooling systems useful in the practice of the invention may include, without limitation: blowing a cold stream of gas, for example, cold air, cold nitrogen or cold helium, onto the surface of the skin (Sturesson and Andersson-Engels (1996) PHYS. MED. BIOL. 41(3): 413-28); spraying a cold liquid stream onto the surface of the skin (Sturesson (1996) supra); conductive cooling using a cold contact surface which does not interfere with the irradiation, for example, a cooled transparent optical material, such as a cooled sapphire tip (see, for example, U.S. Pat. No. 5,810,801); applying a low boiling point, non-toxic liquid, for example, tetrafluoroethane or chlorodifluoromethane, onto the surface of the target tissue, to cool the tissue surface by evaporative cooling, or applying a low boiling point non-toxic liquid onto the surface of the target tissue combined with blowing a stream of gas in the vicinity of the liquid to remove at least a portion of the liquid (U.S. Patent Application No. 20010009997A1).

In a preferred embodiment, cooling is facilitated by a dynamic cooling device (DCD), such as a DCD manufactured by Candela Corp. (Wayland, Mass.). Applications of the DCD have been described in the art and include, for example, Anvari et al. (1996) APPLIED OPTICS 35:33143319; Anvari et al. (1997) PHYS. MED. BIOL. 42:1-18; Ankara et al. (1995) LASERS IN MEDICAL SCIENCE 10:105-112; and Waldorf et al. (1997) DERMATOL. SURG. 23:657-662, U.S. Pat. Nos. 5,820,626 and 5,814,040 and PCT/US97/03449. The DCD provides a timed spray of fluid onto the surface of the skin, prior to, contemporaneous with, and/or after irradiation. Unlike steady-state cooling, for example, an ice cube held against the tissue, dynamic cooling primarily reduces the temperature of the most superficial layers of the skin. For example, it has been estimated that the use of tetrafluoroethane as a cryogen may result in a drop in surface-temperature of about 30-40° C. in about 5-100 ms.

The light delivery system may include an integrated cooling system for cooling the skin surface prior to, contemporaneous with, and/or after irradiation. Accordingly, such a light delivery system would be multi-functional, i.e., capable of both delivering a beam of irradiation and cooling the surface of the skin at the same time. By way of example, an integrated hand piece can be used to apply a beam of light from a laser source and a cryogen spray to a preselected region of the skin surface. Application of the light (heat energy) together with surface cooling can be used to limit thermal injury to the specific portions of the dermis, for example, where the capillaries of the superficial horizontal plexus, are present in a psoriatic plaque, while preserving the epidermis. The hand piece may further include a guide or other measuring device to ensure that the hand piece is positioned at the appropriate height above the surface of the skin to ensure that the beam of radiation and cryogen spray both contact the skin surface at the preselected region. Exemplary hand pieces useful in the practice of the invention are described in U.S. Patent publication no. 20010041886A1.

In another embodiment, the light delivery and cooling systems may comprise separate systems. The cooling system may comprise a container of a cold fluid. Cooling the surface of the skin can be accomplished by applying the cold fluid onto the skin which then extracts heat from the skin on contact. In such an embodiment, a light delivery system comprises, for example, a hand piece containing optics for directing, collimating or focusing the irradiation beam onto the targeting region of the skin surface. The light beam can be carried from the energy source, for example, a laser, to the hand piece by, for example, an optically transparent fiber, for example, an optical fiber. Coolant from a separate reservoir can be applied to the surface of the targeted region. In this embodiment, coolant from the reservoir flows to a dispensing unit separate from the energy delivery system via tubing connecting the reservoir and the dispensing unit. The coolant, once dispensed, can be retained in situ on the surface of the targeted region by a ring, for example, a transparent ring, which can be attached to the energy delivery system.

The preselected region can be cooled prior to, contemporaneous with, and even after the application of the beam of irradiation. The relative timing of cooling the skin surface and the application of irradiation depends, in part, on the depth to which thermal injury is to be prevented. Longer periods of cooling prior to the application of irradiation allow more time for heat to diffuse out of the skin tissue and cause a thicker layer of tissue to be cooled, as compared to the thickness of the layer cooled by a short period of cooling. This thicker layer of cooled skin tissue sustains less thermal injury when the irradiation (heating energy) is subsequently applied. Continued cooling of the skin surface during the delivery of heating energy extracts heat from the upper layers of the skin as heat is deposited, thereby further protecting the upper skin layers (e.g., epidermis and dermis overlaying the target region) from thermal injury.

Selective heating of dermal regions containing the capillary loops of the superficial horizontal plexus of the psoriatic plaque, for example, can be achieved by selecting the appropriate heating and cooling parameters. For example, by choosing the appropriate wavelength it is possible to selectively heat portions of the dermis to a desired depth. Furthermore, it is possible to prevent damage to the skin surface by applying the types of cooling discussed hereinabove. By choosing appropriate parameters for the heating and cooling steps it is possible to selectively heat and thus selectively damage particular zones (target regions) within the skin that contain the capillary loops of the superficial horizontal plexus.

Pre- and Post-treatment Considerations

It is contemplated, that the efficacy of the treatment may be improved by descaling the diseased tissue prior to irradiation. For example, in the case of psoriasis, psoriatic scales may be removed by chemical and/or physical processes well known in the art. During a physical process, the psoriatic scales may be removed, for example, by dermabrasion. This can be accomplished, for example, by application of a 20,000 rpm diamond wheel following local anesthesia with 1% lidocaine and application of a dichlorotetrafluroethane spray (Bjerring et al. (1997) supra). Alternatively, the psoriatic scales may be removed chemically by application of a descaling agent, for example, salicylic acid. This can be accomplished by applying a 5% salicylic acid preparation to the region of interest on a daily basis for a period of one week before irradiation. Other chemical treatments include, for example, the topical application of a drug, for example, a topical corticosteroid to the site of the lesion.

Although the method produces a long lasting effect and, therefore, reduces the number of treatments necessary, it is contemplated that the process may be repeated, for example, every two to about six weeks, or until the disease has been effectively treated. In the case of psoriasis, the treatment is considered to be effective if it reduces the surface area, thickness, or coloration of the psoriatic lesion, or reduces the amount of scaling at the site of the psoriatic lesion.

In light of the foregoing general discussion, the specific examples presented below are illustrative only and are not intended to limit the scope of the invention. Other generic and specific configurations will be apparent to those persons skilled in the art.

### EXAMPLE 1

This example provides one approach using a topically applied pro-photosensitizer for treating psoriasis in a human.

An oil-in-water emulsion is prepared by mixing 5-aminolevulinic acid with GLAXAL™ Base (Shire Canada, Inc., Ontario) to produce a final concentration of 20% (wt/wt) 5-aminolevulinic acid. The emulsion then is applied to a psoriatic lesion on a patient to give a dosage of approximately 25 mg/cm². The treated lesion then is covered with a light-blocking occlusive dressing for about 3 to 14 hours.

The dressing then is removed and excess emulsion removed from the lesion surface. The lesion then is treated with contiguous, minimally overlapping pulses from a pulsed dye laser operating at 585 nanometers, with an irradiated spot size of 10 mm in diameter, pulse duration of 0.45 milliseconds, and fluence of 8 J/cm². The procedure can be repeated at 2 to 6 week intervals, or until the psoriatic lesion is cleared.

### EXAMPLE 2

This example provides another approach for treating psoriasis in which a pro-photosensitizer is applied topically to a previously descaled region.

In this approach, a preparation of 5% salicylic acid in petrolatum is applied twice daily to a psoriatic plaque for about one week prior to irradiation to remove scales overlying the plaque. Afterwards, a solution containing 2% 5-aminolevulinic acid (wt/vol) in water is iontophoresed into the de-scaled plaque using a standard commercial iontophoresis device such as the Phoresor II (Iomed, Salt Lake City, Utah). Approximately 3 hours after iontophoresis, the lesion is treated with contiguous, minimally overlapping pulses from a pulsed dye laser operating at 585 nanometers, with a 10 mm irradiated spot size, pulse duration of 0.45 milliseconds, fluence of 8 J/cm² and dynamic cooling of the epidermal surface. The iontophoresis and irradiation procedures then are repeated at 2 to 6 week intervals or until the psoriatic lesion is cleared.

### EXAMPLE 3

This example provides another approach in which a photosensitizer is applied to a previously descaled psoriatic lesion.

In this approach, a preparation of 5% salicylic acid in petrolatum is applied twice daily to the psoriatic plaque for about one week prior to irradiation to remove the scales overlying the plaque. A chloroform solution of egg phosphatidylcholine (Avanti Polar Lipids, Inc., Alabaster, Ala.) is mixed with hypericin (Sigma Chemical Co., St. Louis, Mo.) dissolved in methanol, and the mixture dried to a thin film using a stream of purified nitrogen gas. Traces of solvent then are removed by vacuum at room temperature for 2 hours. Sterile isotonic saline previously purged with nitrogen is added to the lipid/hypericin mixture, and the resulting mixture shaken to form a homogenous suspension. The mixture then is allowed to stand for 2 hours for further hydration. During preparation of the liposomal formulation of hypericin, exposure of the formulation to light is minimized. The resulting liposome composition comprises as a molar ratio 95.4 egg phosophatidylcholine: 4.6 hypericin.

The liposomal formulation then is applied to the psoriatic plaque to form a layer covering the plaque and a surrounding margin of normal-appearing skin, and covered with a light-protective occlusive dressing. After a period of 1 to 6 hours, the dressing and remaining liposomal formulation is removed from the plaque. The lesion then is treated with contiguous, minimally overlapping pulses from a pulsed dye laser operating at 595 nanometers, with a 10 mm irradiated spot size, pulse duration of 1.5 milliseconds and fluence of 14 J/cm². The skin surface is cooled during irradiation by the application of cold air. The procedure may then be repeated at 2 to 6 week intervals, or until the psoriatic lesion is cleared.

### EXAMPLE 4

This example provides an approach in which a pro-photosensitizer is administered systemically to a patient for treatment of psoriatic lesions.

In this approach, 5-aminolevulinic acid is dissolved in orange juice. The resulting formulation then is consumed by the patient to provide an oral dose of 10 to 20 mg/kg 5-aminolevulinic acid body weight. Between 2 to about 8 hours after consumption, the psoriatic lesions on the patient are treated with contiguous, minimally overlapping pulses from a pulsed dye laser operating at 585 nanometers, with a 10 mm irradiated spot size, pulse duration of 0.45 milliseconds, and fluence of 8 J/cm². The procedures may be repeated at 2 to 6 week intervals, or until the psoriatic lesion is cleared.

### Incorporation By Reference

The disclosure of each of the patent documents and scientific articles referred to herein is incorporated by reference herein.

### Equivalents

The invention may be embodied in other specific forms without departing form the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

What is claimed is:

1. A method of treating a T-cell mediated skin disorder in a pre-selected region of a mammal, the method comprising:
(a) administering to the mammal, an amount of a photosensitizer or a pro-photosensitizer sufficient to permit an effective amount of photosensitizer to localize within the region; and
(b) delivering to the region a beam of pulsed or scanned radiation sufficient to induce selective photothermolysis of blood vessels disposed within the region and to activate the photosensitizer, thereby to ameliorate a symptom of the disorder.

2. The method of claim 1, wherein the disorder is psoriasis or atopic dermatitis.

3. The method of claim 2, wherein the disorder is psoriasis.

4. The method of claim 1, wherein in step (a) the photosensitizer is benzoporphyrin derivative monoacid, hypericin, or rose bengal.

5. The method of claim 1, wherein in step (a) the pro-photosensitizer is 5-aminolevulinic acid or aminolevulinic acid-methyl ester.

6. The method of claim 1, wherein in step (b) the radiation is coherent radiation.

7. The method of claim 6, wherein in step (b) the beam of coherent radiation is a beam of pulsed laser radiation of wavelength from 460 nanometers to 620 nanometers, fluence from 1 to 120 joules per square centimeter per pulse, pulse duration from about 1 microsecond to about 100 milliseconds per pulse, and spot size from 1 millimeter to 30 millimeters in diameter.

8. The method of claim 7, wherein in step (b) the wavelength is from 500 nanometers to 610 nanometers.

9. The method of claim 8, wherein in step (b) the wavelength is from 580 nanometers to 600 nanometers.

10. The method of claim 7, wherein in step (b) the fluence is from 2 to 90 joules per square centimeter per pulse.

11. The method of claim 10, wherein in step (b) the energy is from 4 to 20 joules per pulse.

12. The method of claim 7, wherein in step (b) the spot size is from 5 millimeters to 20 millimeters in diameter.

13. The method of claim 7, wherein in step (b) the pulse duration is from 10 microseconds to 20 milliseconds per pulse.

14. The method of claim 13, wherein in step (b) the pulse duration is from 100 microseconds to 10 milliseconds per pulse.

15. The method of claim 1, wherein in step (a) the photosensitizer or pro-photosensitizer is administered systemically.

16. The method of claim 1, wherein in step (a) the photosensitizer or pro-photosensitizer is administered topically.

17. The method of claim 1, wherein in step (b) the radiation is sufficient to induce purpora in the region.

18. The method of claim 1, whereupon after step (b), the method reduces the surface area, thickness, redness, or scaling of a lesion in the region.

19. The method of claim 1, wherein the combination of steps (a) and (b) is more effective at ameliorating the symptom of the disorder than a method in which no photosensitizer or pro-photosensitizer is administered to the mammal.

20. The method of claim 1, wherein in step (b) the radiation, if delivered in the absence of a photosensitizer or a pro-photosensitizer, would be sufficient to cause selective photothermolysis of blood vessels disposed within in the region.

21. A method of treating a psoriatic lesion in a pre-selected region of a mammal, the method comprising:
(a) administering to the mammal, an amount of a photosensitizer or a pro-photosensitizer sufficient to permit an effective amount of photosensitizer to localize within the region; and
(b) delivering to the region a beam of pulsed or scanned laser radiation sufficient to induce selective photothermolysis of blood vessels disposed within the region and to activate the photosensitizer, thereby to ameliorate a symptom of the lesion.

22. The method of claim 21, wherein in step (a) the photosensitizer is benzoporphyrin derivative monoacid, hypericin, or rose bengal.

23. The method of claim 21, wherein in step (a) the pro-photosensitizer is 5-aminolevulinic acid or aminolevulinic acid-methyl ester.

24. The method of claim 21, wherein in step (b) the beam of laser radiation has a wavelength from 460 nanometers to 620 nanometers, fluence from 1 to 120 joules per square centimeter per pulse, pulse duration from about 1 microsecond to 100 milliseconds per pulse, and spot size from 5 millimeter to 20 millimeters in diameter.

25. The method of claim 24, wherein in step (b) the wavelength is from 580 nanometers to 600 nanometers.

26. The method of claim 24, wherein in step (b) the fluence is from 2 to 90 joules per square centimeter per pulse.

27. The method of claim 24, wherein in step (b) the pulse duration is from 100 microseconds to 10 milliseconds per pulse.

28. The method of claim 21, wherein in step (a) the photosensitizer or pro-photosensitizer is administered systemically.

29. The method of claim 21, wherein in step (a) the photosensitizer or pro-photosensitizer is administered topically.

30. The method of claim 21, wherein in step (b) the radiation is sufficient to induce purpora in the region.

31. The method of claim 21, whereupon after step (b), the method reduces the surface area, thickness, redness, or scaling of the lesion.

32. The method of claim 21, wherein in step (b) the radiation, if delivered in the absence of a photosensitizer or a pro-photosensitizer, would be sufficient to cause selective photothermolysis of blood vessels disposed within the region.

33. The method of claim 21, further comprising the additional step of prior to step (b), removing psoriatic scales from the region.

34. The method of claim 33, wherein the psoriatic scales are removed by abrasion or by application of a descaling agent.
